(19) United States
(12) Plant Patent Application Publication
(10) Pub. No.: US 2006/0143760 P1
(43) Pub. Date: Jun. 29, 2006

(54) CHRYSANTHEMUM PLANT NAMED 'DORA'

(50) CHRYSANTHEMUM MORIFOLIUM; CHRYSANTHEMUM

(75) Inventor: Mark Roland Boeder, The Hague (NL)

Correspondence Address: STEPTOE & JOHNSON LLP, 1330 CONNECTICUT AVENUE, N.W., WASHINGTON, DC, 20036, US

(73) Assignee: Chrysanthemum Breeders Association N.V., Aalsmeer (NL)

(21) Appl. No.: 11/020,165

(22) Filed: Dec. 27, 2004

(51) Int. Cl. A01H 5/00

(52) U.S. Cl. PLT/298

(57) ABSTRACT

A chrysanthemum plant named ‘Dora’ characterized by its small sized blooms with red ray florets and prolific branching; natural season flower date October 18-25; blooming for a period of 4 weeks.

### BACKGROUND OF THE INVENTION

‘Dora’ is a product of a breeding and selection program for outdoor pot mums (garden mums) which had the objective of creating new chrysanthemum cultivars with a daisy type flower, a natural season flower date around October 18-25; blooming for a period of 4 weeks. The new plant of the present invention comprises a new and distinct cultivar of chrysanthemum plant ‘Dora’ is a seedling resulting from a crossing program, which was set up by a previous breeder, and which records are unknown to the inventor. The new and distinct cultivar was discovered and selected as a flowering plant by Mark Roland Boeder on a cultivated field in Rijsenhout, The Netherlands in 2001. The first act of asexual production of ‘Dora’ was accomplished when vegetative cuttings were taken from the initial selection in 2001 in a controlled environment in Rijsenhout, The Netherlands, and propagated further at this location. The new cultivar has been found to retain its distinctive characteristics through successive propagations.

### BRIEF DESCRIPTION OF THE DRAWINGS

The present invention of a new and distinct variety of chrysanthemum is shown in the accompanying drawings, the color being as nearly true as possible with color photographs of this type.

FIG. 1 shows a plant of the cultivar in full bloom.

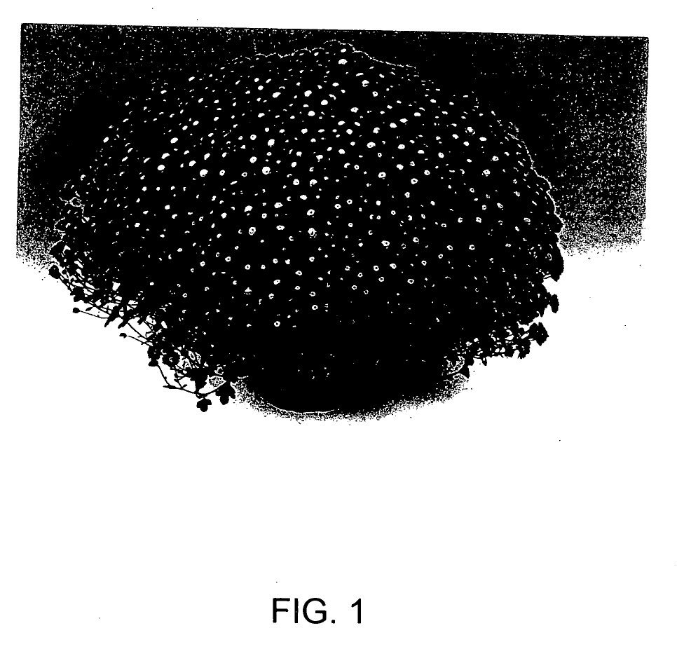

FIG. 2 shows the various stages of bloom of the new cultivar.

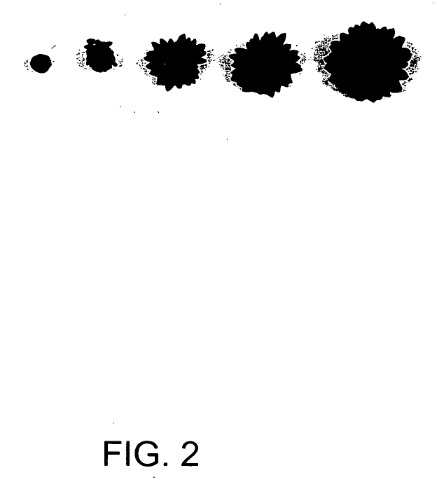

FIG. 3 shows the foliage of the new cultivar.

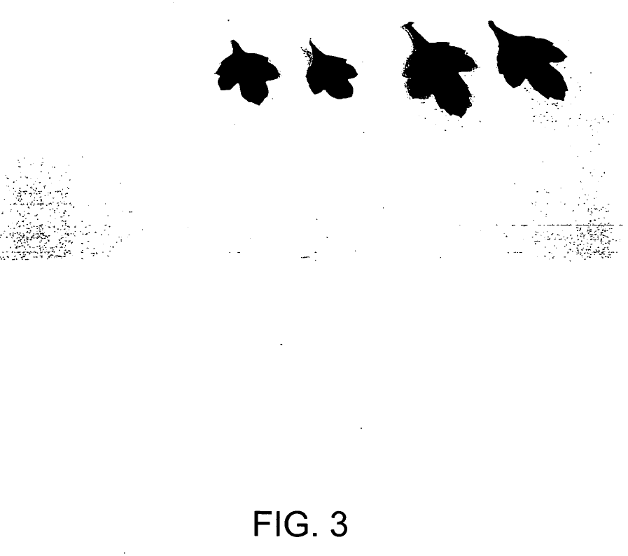

### DESCRIPTION OF THE INVENTION

This new variety of chrysanthemum is of the botanical classification Chrysanthemum morifolium L. The observations and measurements were gathered from plants grown out door in Rijsenhout, The Netherlands under natural day length and temperature and planted week 24 in 2004. The natural blooming date of this crop was October 18-25 (week 43). The average height of the plants was 40 cm. No growth retardants were used. No tests were done on disease or insect resistance or susceptibility. No tests were done on cold or drought tolerance. This new variety produces small sized blooms with red ray florets and a yellow center blooming for a period of 4 weeks.

From the cultivars known to inventor the most similar existing cultivar in comparison to ‘Dora’ are ‘Plutores’ (U.S. Plant Pat. No. 12,919) and ‘Cecaesar’ (U.S. Plant Patent pending). When ‘Plutores’, ‘Cecaesar’ and ‘Dora’ are being compared the following differences are noticed: The differences of ‘Plutores’, ‘Cecaesar’ and ‘Dora’ are (1) Bloom type. The flowers of ‘Plutores’ and ‘Cecaesar’ are decorative, while those of ‘Dora’ are of daisy type. (2) Flower size. The flowers of ‘Dora’ are smaller in size than those of ‘Plutores’ and ‘Cecaesar’ (3) Color ray-florets. The red color of the ray-florets is differently shaded in the three varieties ‘Dora’, ‘Plutores’ and ‘Cecaesar’.

The following is a description of the plant and characteristics that distinguish ‘Dora’ as a new and distinct variety. The color designations are taken from the plant itself. Accordingly, any discrepancies between the color designations and the colors depicted in the photographs are due to photographic tolerances. The color chart used in this description is: The Royal Horticultural Society Colour Chart, edition 1995.

TABLE 1
Botanical Description of chrysanthemum plant ‘DORA’

| | |
|---|---|
| Bud | |
| Size | Small; cross-section 0.3 cm, |
| | height 0.2 cm |
| Outside Color | Greyed-purple 187B |
| Involucral bracts | 2 rows, length 7 mm, width |
| | 3 mm |
| Involucral bracts among disc-florets | Not present |
| Involucral bracts color | Green 139C |
| Bloom | |
| Type | Daisy |
| Height | 1 cm |
| Size | Small |
| Fully Expanded | 3 cm |
| Peduncle length | 3.5-4 cm |
| Peduncle color | Yellow-green 144A to Green |
| | 138A |
| Number of blooms per branch | Approx. 5-7 blooms per |
| | branch |
| Performance on the plant | 4 weeks |
| Seeds | Produced in small quantities, |
| | ovate grey-brown 199A, |
| | 1½ mm in length. |
| Fragrance | Typical chrysanthemum, |
| | slightly |
| Color | |
| Center of the flower (disc-florets) | Immature Yellow-green 145A |
| | Mature Yellow-orange 15A |
| Color of upper surface of the ray- | Greyed-red 178B |
| florets | |
| Color of the lower surface of the | Greyed-orange 175D |
| ray-florets | |
| Tonality from Distance | A garden mum with red |
| | flowers and a yellow disc |
| Discoloration to color | Greyed-red 178B |
| Ray florets | |
| Texture | Upper and under side smooth |
| Number | 70-80 |
| Cross-section | Flat |
| Longitudinal axis of majority | Straight |
| Length of corolla tube | 0.3 cm |
| Ray-floret margin | Entire |
| Ray-floret length | 1.3 cm |
| Ray-floret width | 0.3 cm |
| Ratio length/width | High |
| Shape of tip | Pointed |
| Disc florets | |
| Disc diameter | 1 cm |
| Distribution of disc florets | Numerous, clearly visible at |
| | all stages |
| Shape | Tubular |
| Receptacle shape | Conical raised |
| Reproductive Organs | |
| Stamen | Present in disc florets only |
| Stamen color | Yellow-green 144A |
| Pollen | Present |
| Pollen color | Yellow 7A |
| Styles | Thin |
| Style color | Yellow 13A |
| Style Length | 3 mm |
| Stigma color | Yellow-green 144A |
| Stigma Width | 1 mm |
| Ovaries | Enclosed in calyx |
| Plant | |
| Form | Grown as a spray type |
| | potmum, outdoor mounded |
| | and round |
| Growth habit | Spherical shape |
| Growth rate | Medium vigor |
| Height | 40 cm |
| Width | 55 cm |
| Stem Color | Greyed-brown 199B |
| Stem Strength | Strong |
| Stem Brittleness | Not brittle |
| Stem Anthocyanin Coloration | Absent |
| Internode length | 1.5-3 cm |
| Length of lateral branch | From top to bottom 13 cm |
| Lateral branch color | Yellow-green 144B |
| Lateral branch, attachment | Medium strength |
| Branching (average number of | Prolific with 8-10 breaks |
| lateral branches) | after pinching |
| Natural season blooming date | October 18-25 |
| Foliage | |
| Leaf color | Upper side Green 137A |
| | Lower side Green 138A |
| Color midvein | Upper side Green 138C |
| | Lower side Green 139D |
| Size | Small; length 2-3.5 cm, |
| | width 2-2.5 cm |
| Quantity (number per lateral | 18-20 |
| branch) | |
| Shape | Ovate |
| Texture upper side | Glabrous |
| Texture under side | Pubescent |
| Venation arrangement | Palmate |
| Shape of the margin | Serrated |
| Shape of Base of Sinus Between | Rounded |
| Lateral Lobes | |
| Margin of Sinus Between Lateral | Diverging |
| Lobes | |
| Shape of Base | Truncate |
| Apex | Mucronulate |
| Petiole length | 0.5 cm |
| Petiole color | Green 138C |

TABLE 2
Differences with the comparison varieties

| | ‘Dora’ | ‘Plutores’ | ‘Cecaesar’ |
|---|---|---|---|
| Bloom type | Daisy | Decorative | Decorative |
| Flower size | 3 cm | 4.5 cm | 5.5 cm |
| Color upper | Greyed-red 178B | Red 46A | Greyed-red 178D |
| side ray-florets | | | at tip and Yellow |
| | | | 7C at base |

I claim:

1. A new and distinct variety of chrysanthemum plant as described and illustrated.
